(19) United States
(12) Patent Application Publication
(10) Pub. No.: US 2022/0347086 A1
(43) Pub. Date: Nov. 3, 2022

(54) COMPOSITION AND APPLICATION THEREOF IN PREPARATION OF SKINCARE PRODUCTS FOR NIGHT OWLS

(71) Applicant: SYoung Cosmetics Manufacturing Co., Ltd., Changsha (CN)

(72) Inventors: Yuefeng DAI, Changsha (CN); Shaowei YAN, Changsha (CN); Jingru QIAN, Changsha (CN); Xiaoqun XU, Changsha (CN)

(21) Appl. No.: 17/763,918

(22) PCT Filed: Aug. 31, 2020
(86) PCT No.: PCT/CN2020/112457

§ 371 Date: Mar. 25, 2022

(30) Foreign Application Priority Data
Sep. 26, 2019 (CN) 201910918246.X

(51) Int. Cl. A61K 8/9794; A61K 8/73; A61K 8/60; A61K 8/49; A61K 8/46; A61K 8/34; A61Q 19/00

(52) U.S. Cl. CPC A61K 8/9794; A61K 8/735; A61K 8/606; A61K 8/4946; A61K 8/466; A61K 8/345; A61Q 19/00

(57) ABSTRACT

Relating to the technical field of skincare products, particularly to a composition and an application thereof in preparation of skincare products. The provided composition comprises adenosine, Leucojum aestivum bulb extract, sodium hyaluronate, skin conditioner A containing glycoprotein and amino acids, and skin conditioner B containing glutaminoethyl imidazole. The components cooperate with one another and promote one another, thereby achieving good effects of hydration and moisturizing, restoring skin glow, and promoting cell energy synthesis. Using the composition in the preparation of cosmetics can ameliorate a series of skin problems of night owls.

### TECHNICAL FIELD

The present disclosure relates to the technical field of skincare products, and in particular, to a composition and an application thereof in preparation of skincare products for night owls.

### BACKGROUND

In modern society, due to various factors of life and work, people fall asleep later and later. According to the “2018 China Sleep Index” report released by the Sleep Medicine Professional Committee of the Chinese Medical Doctor Association, ¾ of the “post-90s” fall asleep after 11 pm, and ⅓ thereof fall asleep at 1 am, and more than 60% of them have an owl-type (sleep late and get up late) daily routine and a hummingbird type (sleep late and get up early) daily routine. Often staying up late will not only lead to decreased resistance and memory loss, but also directly manifest its adverse effect on the skin.

Skin problems caused by staying up late mainly include:

1. Excessive secretion of skin oil: Staying up late will affect the normal endocrine of the human body, resulting in disorder of skin metabolism, with a manifestation of stimulation of sebaceous glands. As a result, a large amount of oil is produced. If the skin is not cleaned timely, it is easy to clog the pores due to excess oil, resulting in the formation of acne and pimples.

2. Dry skin: When the sebaceous glands of the skin function normally, the sebum secreted by the sebaceous glands covers the surface of the skin, thereby reducing the water loss of the skin. When the endocrine disorder after staying up late for a long time causes the skin to secrete less sebum, the skin will lose more water and the water content of the skin will decrease faster, resulting in dry skin.

3. Rough skin: Late night is an important stage for the skin to repair itself. However, when people stay up late, the energy originally used to maintain the cells for metabolism, self-repair, and renewal is consumed by the mechanism of staying up late, and the body does not have more energy to participate in cell repair. As a result, skin aging is accelerated and the skin more likely becomes rough.

4. Dull complexion: Due to the lack of timely self-repair and renewal of skin cells, the cells die. During the process of cell death, some inflammatory factors will be released. The inflammatory factors will activate melanocytes and the melanocytes can produce melanin, eventually leading to skin pigmentation. After death of skin cells, if the skin cells are not renewed in time, the tissue structure of the skin will be damaged, which will cause the loss in skin glow and cause dull complexion.

5. Dark circles and eye puffiness

Night is the physiological rest time of the human body, and it is also an important time for the repair and metabolism of various bodies. Staying up late causes disorder in the repair and metabolism of the body. The metabolized products cannot be eliminated from the body in time, and the metabolites in the blood increase. The local blood circulation is not smooth, causing dark circles and heavy bags under the eyes in the orbital area where the skin is relatively thin.

At present, there are few types of skincare products for night owls. Some compositions focus on hydrating and moisturizing or promoting blood circulation and speeding up metabolism; while other compositions use Chinese herbal extracts as active ingredients, but their specific efficacies are not clear. It can be seen that the current limited products used for caring the skin of night owls or local skin such as orbits are not based on the mechanism of staying up late on skin damage.

### SUMMARY

In view of this, the technical problem to be solved by the present disclosure is to provide a composition and an application thereof in preparation of skincare products for night owls. The composition of the present disclosure has good hydrating and moisturizing effect and can restore skin glow and promote cell energy synthesis.

The composition of the present disclosure includes the following components in parts by mass:

0.5 to 2.0 parts of a Leucojum aestivum bulb extract;

0 to 0.5 part of sodium hyaluronate;

0.2 to 1.0 part of a skin conditioner A;

0.2 to 2.0 parts of a skin conditioner B; and

0.1 to 2.0 parts of adenosine.

The skin conditioner A includes, in percentage by mass:

3% of yeast glycoprotein;

3% of glutamate;

0.55% of valine;

0.55% of threonine;

1.1% of a preservative;

0.05% of a stabilizer; and

the balance of water.

The skin conditioner B includes, in percentage by mass:

1% of glutamyl aminoethyl imidazole;

0.4% of a preservative; and

the balance of water.

The preservative in the skin conditioner A includes phenoxyethanol and ethylhexylglycerol at a mass ratio of 1:1.

The stabilizer in the skin conditioner A is sodium metabisulfite.

The preservative in the skin conditioner B includes sorbic acid and phenoxyethanol at a mass ratio of 1:3.

In the present disclosure, the sodium hyaluronate includes the following components in percentage by mass:

14.6% of sodium hyaluronate with a molecular weight of 2.1 million;

23.6% of sodium hyaluronate with a molecular weight of 1.3 million; and

61.8% of hydrolyzed sodium hyaluronate.

In some embodiments, the composition includes the following components in parts by mass:

1.0 part of the Leucojum aestivum bulb extract;

0.1 part of the sodium hyaluronate;

0.5 part of the skin conditioner A;

0.5 part of the skin conditioner B; and

0.2 part of the adenosine.

In some embodiments, the composition includes the following components in parts by mass:

0.5 part of the Leucojum aestivum bulb extract;

0.2 part of the skin conditioner A;

0.2 part of the skin conditioner B; and

0.1 part of the adenosine.

In some embodiments, the composition includes the following components in parts by mass:

2.0 parts of the Leucojum aestivum bulb extract;

0.5 part of the sodium hyaluronate;

1.0 part of the skin conditioner A;

2.0 parts of the skin conditioner B; and

2.0 parts of the adenosine.

The disclosure takes the skin state of night owls as the research object, and prepares the composition with the biological rhythm mechanism of the skin and skin repair as the background. In terms of biological rhythm adjustment of skin: the skin conditioner B (containing biological rhythm peptide) is used to regulate the skin's biological clock gene, promote the repair and proliferation of keratinocytes, promote the synthesis of vitamin D, and activate the conversion of vitamin D to activate the skin's barrier capacity. The skin conditioner B rich in glycoprotein, glutamic acid, valine, and threonine is adopted to promote sugar degradation and mitochondrial respiration, ensuring that the ATP (adenosine 5′-triphosphate) of skin cells is at a high level throughout the day, and sufficient energy is supplied to cells during the day for defending against external adverse factors, and ensuring sufficient energy supply when skin cells need to be repaired at night. Moreover, by addition of adenosine, energy needed by cells can be directly replenished. In terms of skin repair: by addition of hyaluronic acid (including hyaluronic acid with a molecular weight of 2.1 million, hyaluronic acid with a molecular weight of 1.3 million, and hydrolyzed hyaluronic acid) and adenosine, a combination for hydrating and moisturizing skin from the three dimensions of cell repair, stratum corneum hydration, and epidermal hydration is formulated; moreover, the addition of the Leucojum aestivum bulb extract with a powerful cell repairing effect can inhibit the proliferation of melanocytes, reduce the production of melanin, and avoid the dull skin caused by melanin deposition. The components in the composition of the present disclosure cooperate with each other and promote each other, thus achieving a good synergistic effect.

Application of the composition of the present disclosure in preparation of skincare products for night owls is disclosed.

The skincare products have functions of hydrating and moisturizing, restoring skin glow, and promoting cell energy synthesis.

The present disclosure further provides a skincare product for night owls, including the composition of the present disclosure.

In the skincare product of the present disclosure, the composition has a percentage of 1% to 7.5% by mass.

In some specific embodiments, the composition has percentages of 1%, 2.3%, and 7.5% by mass.

The skincare product of the present disclosure includes:

1% to 7.5% of the composition of the present disclosure;

9% of a moisturizer;

1% of a penetration enhancer;

0.4% of a cuticle softener;

1% of a preservative; and

the balance of water.

In the skincare product of the present disclosure,

the moisturizer includes glycerin and 1,3 butanediol at a mass ratio of 1:1;

the penetration enhancer is pentanediol;

the cuticle softener is hydroxyethylpiperazine ethanesulfonic acid;

the preservative is PHL.

The skincare product of the present disclosure includes the following components in percentage by mass:

0.5% to 2.0% of the Leucojum aestivum bulb extract;

0 to 0.5% of sodium hyaluronate;

0.2% to 1.0% of the skin conditioner A;

0.2% to 2.0% of the skin conditioner B;

0.1% to 2.0% of adenosine;

8% of glycerol;

1% of 1,3 butanediol;

1% of pentanediol;

0.4% of hydroxyethylpiperazine ethanesulfonic acid;

1% of PHL; and

the balance of water.

The skin conditioner A includes, in percentage by mass:

3% of yeast glycoprotein;

3% of glutamate;

0.55% of valine;

0.55% of threonine;

1.1% of a preservative;

0.05% of a stabilizer; and

the balance of water.

The skin conditioner B includes, in percentage by mass:

1% of glutamyl aminoethyl imidazole;

0.4% of a preservative; and

the balance of water.

In some embodiments, the skincare product includes the following components:

1.0% of the Leucojum aestivum bulb extract;

0.1% of the sodium hyaluronate;

0.5% of the skin conditioner A;

0.5% of the skin conditioner B;

0.2% of the adenosine;

8% of the glycerol;

1% of the 1,3 butanediol;

1% of the pentanediol;

0.4% of the hydroxyethylpiperazine ethanesulfonic acid;

1% of the PHL; and

the balance of water.

In some embodiments, the skincare product includes the following components:

0.5% of the Leucojum aestivum bulb extract;

0.2% of the skin conditioner A;

0.2% of the skin conditioner B;

0.1% of the adenosine;

8% of the glycerol;

1% of the 1,3 butanediol;

1% of the pentanediol;

0.4% of the hydroxyethylpiperazine ethanesulfonic acid;

1% of the PHL; and

the balance of water;

In some embodiments, the skincare product includes the following components:

2.0% of the Leucojum aestivum bulb extract;

0.5% of the sodium hyaluronate;

1.0% of the skin conditioner A;

2.0% of the skin conditioner B;

2.0% of the adenosine;

8% of the glycerol;

1% of the 1,3 butanediol;

1% of the pentanediol;

0.4% of the hydroxyethylpiperazine ethanesulfonic acid;

1% of the PHL; and

the balance of water.

The skincare product of the present disclosure includes toners, lotions, creams, facial masks, essences, gels and the like.

A method of preparing the skincare product of the present disclosure, including:

adding hydroxyethylpiperazine ethanesulfonic acid, sodium hyaluronate, and adenosine to deionized water, heating to 75° C. to 80° C., and stirring and dissolving at 250 r/min to 300 r/min for 20 min;

adding glycerol, 1,3-butanediol, and pentanediol, and stirring and dissolving at 300 r/min for 15 min with a temperature maintaining at 65° C. to 75° C.; and

adding a Leucojum aestivum bulb extract, a skin conditioner A, a skin conditioner B, and PHL, and stirring at 100 r/min for 15 min with a temperature maintaining at 35° C. to 45° C., and then cooling to room temperature to prepare the skincare product.

The composition of the present disclosure includes adenosine, the Leucojum aestivum bulb extract, sodium hyaluronate, the skin conditioner A containing glycoprotein and amino acids, and the skin conditioner B containing glutaminoethyl imidazole. The components cooperate with one another and promote one another, thereby achieving good effects of hydrating and moisturizing, restoring skin glow, and promoting cell energy synthesis. Using the composition in the preparation of cosmetics can ameliorate a series of skin problems of night owls.

### BRIEF DESCRIPTION OF THE DRAWINGS

FIG. 1 shows the effect of each sample on the amount of energy synthesis of cells;

FIG. 2 shows an instant hydrating effect of each sample;

FIG. 3 shows a long-acting hydrating effect of each sample;

FIG. 4 shows a short-term effect of each sample on water loss of skin;

FIG. 5 shows a long-term effect of each sample on the water loss of skin; and

FIG. 6 shows the effect of each sample on skin glow.

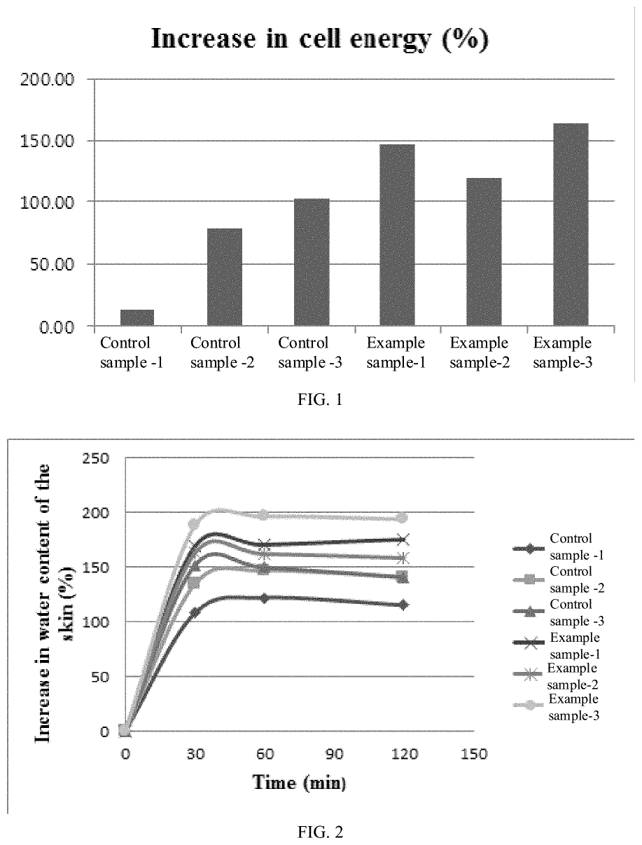

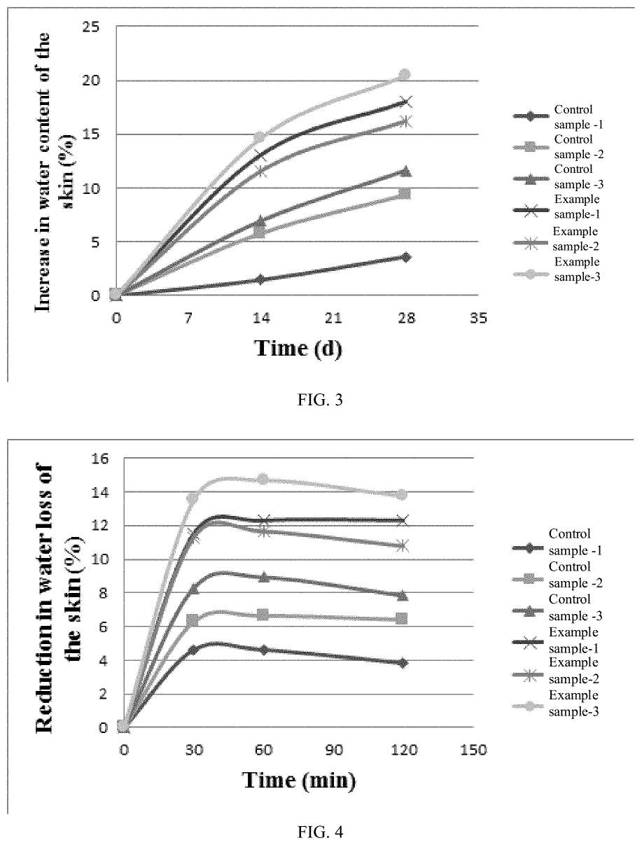

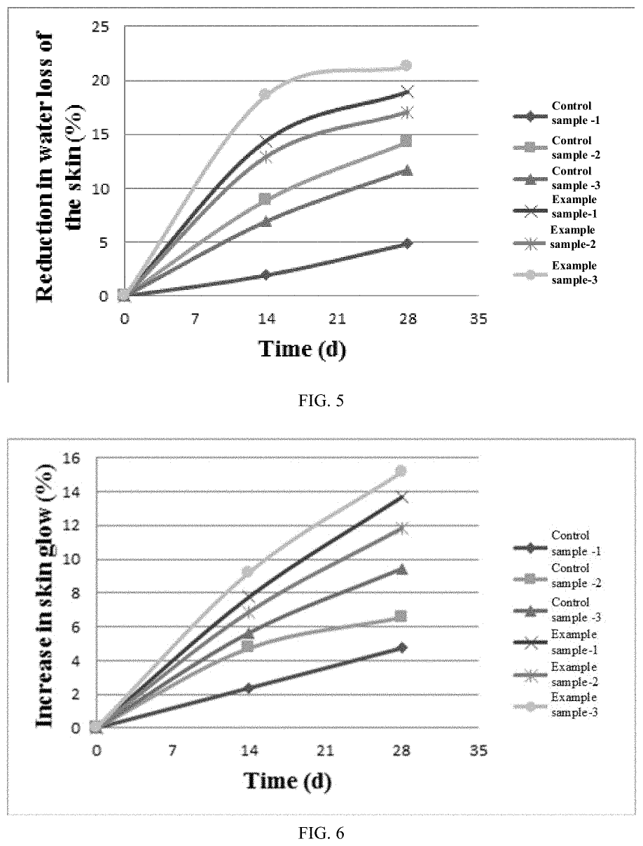

### DETAILED DESCRIPTION OF THE EMBODIMENTS

The present disclosure provides a composition and an application thereof in preparation of skincare products for night owls. Those skilled in the art can learn from the content of the present disclosure and appropriately improve the process parameters to implement the present disclosure. Particularly, it should be pointed out that all similar substitutions and modifications are obvious to those skilled in the art, and they are all deemed to be included in the present disclosure. The methods and applications of the present disclosure have been described by preferred embodiments. It is obvious that relevant persons can make changes or appropriate modifications and combinations of the methods and applications herein without departing from the content, spirit and scope of the present disclosure to implement and apply the technology of the present disclosure.

Test materials adopted in the present disclosure are all common commercial products and can be purchased from the market.

In the following embodiments, the adopted skin conditioner A includes:

3% of glycoprotein;

3% of glutamate;

0.55% of valine;

0.55% of threonine;

0.55% of phenoxyethanol;

0.55% of ethylhexylglycerin;

0.05% of sodium metabisulfite; and

91.75% of water.

The adopted skin conditioner B includes:

1% of glutamyl aminoethyl imidazole;

0.1% of sorbic acid;

0.3% of phenoxyethanol; and

98.6% of water.

The glutamyl aminoethyl imidazole has a structural formula of:

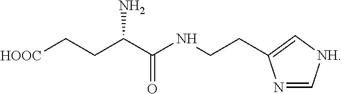 [SMILES: N[C@@H](CCC(=O)O)C(=O)NCCc1c[nH]cn1]

The Leucojum aestivum bulb extract is extracted by using bulbs of Leucojum aestivum in a dormant period as raw materials, and its main active ingredient is abscisic acid. The Leucojum aestivum bulb extract adopted in the present disclosure includes:

1.1% of Leucojum aestivum bulb extract;

0.5% of phenoxyethanol;

0.3% of potassium sorbate;

0.2% of chlorphenesin; and

97.9% of water.

Sodium hyaluronate is also called trimolecular hyaluronic acid and includes:

14.6% of sodium hyaluronate with a molecular weight of 2.1 million;

23.6% of sodium hyaluronate with a molecular weight of 1.3 million; and

61.8% of hydrolyzed sodium hyaluronate.

In the embodiments of the present disclosure, the adopted skin conditioner A, skin conditioner B, and the Leucojum aestivum bulb extract are all purchased from the market, and their trade names are REVITALIN® PF, CHRONOCYCLIN®, and IBR Snowflake® 1001 in sequence.

In conjunction with embodiments, the present disclosure is further elaborated as follows.

### Examples

The formulas of various groups are shown in Table 1:

TABLE 1
Component contents of the formulas of various groups (1%)

| | Group | | | | | |
|---|---|---|---|---|---|---|
| | Control | Control | Control | | | |
| Component | sample 1 | sample 2 | sample 3 | Example 1 | Example 2 | Example 3 |
| Glycerol | 8.0 | 8.0 | 8.0 | 8.0 | 8.0 | 8.0 |
| 1,3 butanediol | 1.0 | 1.0 | 1.0 | 1.0 | 1.0 | 1.0 |
| Pentanediol | 1.0 | 1.0 | 1.0 | 1.0 | 1.0 | 1.0 |
| Hydroxyethylpiperazine | 0.4 | 0.4 | 0.4 | 0.4 | 0.4 | 0.4 |
| ethanesulfonic acid | | | | | | |
| PHL | 1.0 | 1.0 | 1.0 | 1.0 | 1.0 | 1.0 |
| Leucojum aestivum bulb extract | — | 1.0 | 0.5 | 1.0 | 0.5 | 2.0 |
| Sodium hyaluronate | — | 0.1 | 0.60 | 0.1 | 0.05 | 0.5 |
| Skin conditioner B | — | — | 0.1 | 0.5 | 0.2 | 1.0 |
| Skin conditioner A | — | — | 0.1 | 0.5 | 0.2 | 2.0 |
| Adenosine | — | — | 1.00 | 0.2 | 0.1 | 2.0 |
| Water | 88.6 | 87.5 | 86.3 | 86.3 | 87.55 | 81.1 |

Hydroxyethylpiperazine ethanesulfonic acid, sodium hyaluronate, and adenosine were weighed according to a formula ratio and then added with deionized water. The resulting mixture was then heated to 75° C. to 80° C. and stirred at 250 r/min to 300 r/min for 20 min until the hydroxyethylpiperazine ethanesulfonic acid, the sodium hyaluronate, and the adenosine were dissolved and the solution became transparent.

Glycerol, 1,3-butanediol, and pentanediol were added according to the formula ratio and the resulting solution was then stirred at 300 r/min for 15 min with the temperature maintaining at 65° C. to 75° C., until all substances added were dissolved.

The Leucojum aestivum bulb extract, the skin conditioner A, the skin conditioner B, and PHL were weighed and added according to the formula ratio and the resulting solution was then stirred at a speed of 100 r/min for 15 min with a temperature maintaining below 45° C., and then rested still to return to room temperature.

### Efficacy Evaluation

The physiological activity of skin tissue is strictly controlled by biological rhythms. During the day, skin cells mainly work on the natural defenses (UV resistance, anti-pollution, etc.) of the skin. During the night, skin cells focus on cell repair and metabolism. Studies have found that skin's biological clock genes, such as CLOCK and PERIOD-1, are associated with the proliferative activities of keratinocytes, fibroblasts, melanocytes, and the like. Irregular biological rhythms can alter the periodic activation of biological clock genes, resulting in changes in the physiological metabolism of the skin. After the physiological metabolism of the skin changes, the ability of the skin to resist external unfavorable factors will decrease during the day, which will lead to increased damage to the skin. The ability of skin cells to repair themselves at night will become weaker, eventually leading to damage to the skin barrier, with the manifestations of continuous decrease in water content of the skin, increase in water loss, and continuous declination in skin glow. Damage to skin cells will also lead to a continuous decrease in the level of ATP synthesized by cells. The present disclosure evaluates the efficacy of the composition from four aspects: cell energy change, hydrating effect, water loss of skin, and improvement of skin glow.

1) Increase in Cell Energy

Adenosine 5′-triphosphate (ATP) is the most basic carrier of energy exchange in the living body, and the change of its content is directly related to the energy metabolism of various organs. As the most important energy molecule, ATP plays an important role in various physiological and pathological processes of cells. Changes in ATP level can affect cell function. Usually when cells are in apoptosis, necrosis or in some toxic state, ATP level will decrease. The decrease in ATP level during apoptosis usually coincides with a decrease in mitochondrial membrane potential. An ATP assay kit can be used to detect the ATP level in cells.

Experimental equipment: microplate reader, water bath, pipette gun, centrifuge, 96-well plate, and ATP assay kit

Experimental materials: Keratinocytes, Dulbecco's modified eagle medium (DMEM), fetal bovine serum (FBS), and Dulbecco's Phosphate Buffered Saline (DPBS)

Experimental Method:

1. After the keratinocytes were digested, the cells were dispersed in DMEM medium, the cells were counted using a Hemacytometer, and then the cells were diluted using DMEM to a concentration of 5×10⁴ cells/ml.

2. The diluted cell solutions were inoculated onto culture dishes, respectively.

3. The cells were cultured for 24 h in a 37° C., 5% CO₂ incubator.

4. Preparation of samples to be tested: The samples to be tested were diluted with DMEM medium to a concentration of 0.1%, and 10 ml of each sample was prepared.

5. After the cells were cultured for 24 h, it was observed whether the cells grew completely adherently. If the cells grew completely adherently, the original medium was removed and washed with DPBS.

6. After the DPB S was removed, the previously prepared media each containing 0.1% of the sample to be tested were added, respectively.

7. After the addition of the samples, the samples were cultured for 48 h in a 37° C., 5% CO₂ incubator.

Collection and treatment of keratinocytes: After the keratinocytes were collected, the medium was removed by centrifugation, 0.5 ml of double distilled water was added, and the mixture was evenly mixed. The aqueous cell solution was placed in wastewater, heated and boiled for 10 min, vortexed and well mixed for 1 min, and then centrifuged at 4000 rpm for 10 min. The supernatant was taken for testing.

The specific reagents and operating steps are as follows.

Experimental Reagents:

Reagent 1: Substrate liquid 1 which is powder. When in use, the substrate liquid 1 is dissolved with 10 ml of distilled water and heated to be dissolved in boiling water.

Reagent 2: Substrate liquid 2 which is liquid. The substrate liquid 2 can be used directly.

Reagent 3: Accelerator. When in use, the powdery accelerator is added with a diluent to form a solution.

Reagent 4: Precipitant

Reagent 5: Developer. When in use, solution A is added to solution B and the mixed solution is then evenly mixed for later use.

Reagent 6: Terminator

ATP Standard: 1 mM ATP standard solution prepared with DDW.

Preparation of Detection System:

TABLE 2
Detection system

| Reagent name (μL) | Test tube | Control tube | Standard tube | Blank tube |
|---|---|---|---|---|
| Sample | 30 | 30 | — | — |
| 1 mM standard solution | — | — | 30 | 30 |
| Reagent 1 | 100 | 100 | 100 | 100 |
| Reagent 2 | 200 | 200 | 200 | 200 |
| Reagent 3 | 30 | — | 30 | — |
| Distilled water | — | 30 | — | 30 |

Evenly mix them thoroughly to react at 37° C. for 30 min.

| | | | | |
|---|---|---|---|---|
| Reagent 4 | 50 | 50 | 50 | 50 |

Evenly mix the resulting solution thoroughly and centrifuge the solution
at 4000 rpm for 5 min. Take 300 μL of supernate for testing

| | | | | |
|---|---|---|---|---|
| Sample supernate | 300 | 300 | 300 | 300 |
| Reagent 5 | 500 | 500 | 500 | 500 |

Mix the resulting solution well and rest the
resulting solution still for 2 min at room temperature.

| | | | | |
|---|---|---|---|---|
| Reagent 6 | 500 | 500 | 500 | 500 |

The resulting solution was evenly mixed and then rested still for 5 min at room temperature. 200 μL of samples are respectively taken from the test sample tubes, the control tubes, the standard tubes, and the blank tubes to a 96-well plate to test their absorbance at a wavelength of 630 nm.

Therefore, the ATP level of each test sample cell can be obtained as follows:

ATP level (μmol)=[(A test tube−A control tube)/(A standard tube−A blank)]×standard concentration×dilution factor

Increase in cell energy for each test sample:

ATP Increase (%)=(ATPₙ-ATP₀)/ATP₀×100% (where ATPₙ refers to the cell energy of each test sample, and ATP₀ refers to the cell energy of the blank sample)

Experimental Results

TABLE 3
Energy synthesis of cells promoted by the disclosed combination

| | Increase in cell energy (%) |
|---|---|
| Control sample-1 | 12.47 |
| Control sample-2 | 78.35 |
| Control sample-3 | 102.40 |
| Example sample-1 | 146.62 |
| Example sample-2 | 120.37 |
| Example sample-3 | 164.32 |

Experimental results: From the test results of cell energy, it can be seen that the control sample-1 has almost no efficacy of promoting the increase of cell energy. With the addition of substances that can promote the cell energy, example samples 1 to 3 can increase cell energy by 120.37% to 164.32%. The increase of cell energy contributes to the repair of damaged skin conditions. Statistical analysis:

Compared with Comparative Example 1, Examples 1 to 3 can significantly increase the cell energy, indicating that the composition of the present disclosure has a significant efficacy in increasing cell energy; p<0.05.

Compared with Comparative Example 2, Examples 1 to 3 have more significant effect in increasing cell energy, indicating that the composition of the present disclosure is more reasonable in its component matching, each component is indispensable, and all the components work together to produce a significant synergistic effect.

Compared with Comparative Example 3, Examples 1 to 3 have more significant effect in increasing cell energy, indicating that the composition of the present disclosure has a better effect under the same content of active ingredients. In other words, the components in the composition of the present disclosure are in a reasonable and appropriate ratio.

Among the samples, Example 3 has the most significant effect, indicating that its ratio and concentration are the most suitable, and the effect of the obtained sample is significantly different from the effects of other groups; p<0.05.

2) Instant and Long-Acting Hydrating Effect on the Skin
Test equipment: Multifunctional skin tester with a model of Corneometer CM825 MDD provided by CK company, German.
Test principle: The capacitance method is used to test the water content of the stratum corneum of human skin based on the principle that because of the significant difference in the dielectric constant between water and other substances, the measured capacitance values of the skin are different according to the different water content of the skin, and the observed parameters can represent the water values of the skin.
Test environment: The test environment had a temperature of 22±1° C. and a humidity of 50±5%, and the temperature and humidity were dynamically detected in real time.
Test volunteers: There were at least 30 valid volunteers, aged between 16 and 65 years (except pregnant or lactating women) and their basic values of the skin water content measurement significance of the capacitance method of the forearm test area were between 15 and 100. The valid volunteers were: those who have no serious system disease, no immunodeficiency or autoimmune disease; those who have no history of severe allergies to skin care cosmetics in the past; those who have not used hormone drugs and immunosuppressants in the past month; those who have not participated in other clinical trials; those who have used the test drugs as required and have complete information. All the volunteers should fill out an informed consent form before testing.

Test Steps:

Preparation before testing: No products (cosmetics or topical drugs) were allowed to be applied to the test site 2 to 3 days before testing. Before testing, the subjects were willing to clean inner sides of forearms of both hands and let them air dry naturally. After cleaning, the subjects were marked with the measurement areas on the inner sides of the forearms of both hands. Before the formal testing, the subjects were required to sit still in a standard room for at least 30 min without drinking water, with their forearms exposed, taking a test posture and staying relaxed.

Test process: In the experiment, 3×3 cm² test areas were marked on the inner sides of both the left and right arms. Multiple areas could be marked on the same arm, with a spacing of 1 cm. Both the test products and the blank controls were randomly distributed on the left and right arms. The water content measurements were performed on the skin in the test and control areas using a probe Corneometer CM825 MDD. The measurement was carried out 15 times in parallel on each area. The blank value of each test area was measured first, and then the sample was injected into a piece of film cloth according to a dosage of 0.072 ml of sample/cm², and then the film cloth was applied to the test area for 20 min. 20 min later, the mask cloth was removed. 10 min later, the water content of the skin in this area was tested, thus obtaining the water content of the skin at the 30th minute. Then, the water contents of the skin in the test areas and the blank control areas were measured respectively at the 1st hour and the 2nd hour (measured according to these time points during the verification), and the test for the same volunteer was done by the same measurement personnel.

For the long-acting hydrating test, each subject applied the test sample to the same area every day according to the above test method, and on day 14 and day 28, the water content of the skin was tested without applying the test sample.

Test data: According to the experimental design, the water content of the skin in each time period was measured, and the increase in water content of the skin at each time point was calculated.

$$Increase in water content of the skin ( \% ) = \frac{( {M}_{1} - {M}_{0} )}{{M}_{0}} \times 100 \%$$

Experimental Results

2.1) Instant Hydrating Results

TABLE 4
Instant hydrating effect

| | Increase in water content of the skin (%) | | | |
|---|---|---|---|---|
| Time (min) | 0 | 30 | 60 | 120 |
| Control sample-1 | 0.0 | 108.1 | 122.3 | 115.6 |
| Control sample-2 | 0.0 | 134.6 | 147.1 | 141.0 |
| Control sample-3 (inappropriate) | 0.00 | 151.7 | 149.9 | 141.2 |
| Example sample-1 (disclosed sample) | 0.00 | 169.0 | 170.7 | 175.5 |
| Example sample-2 (lower-limit sample) | 0.00 | 162.9 | 162.3 | 158.7 |
| Example sample-3 (upper-limit sample) | 0.00 | 188.0 | 196.5 | 194.0 |

Results: In terms of the instant hydrating effect, the control sample-1, as a basic formula, has a certain hydrating effect, and the increase in water content is 108.1%. For the Example sample-1, the water content of the skin increases to 169.0% at the 30th minute, and the hydrating effect is still about 175.5% at the 120th minute. The instant hydrating effect is obvious and lasts for a long time.

2.2) Long-Acting Hydrating Effect

TABLE 5
Long-acting hydrating effect

| | Increase in water content of the skin (%) | | |
|---|---|---|---|
| Time (d) | 0 | 14 | 28 |
| Control sample-1 | 0.00 | 1.46 | 3.62 |
| Control sample-2 | 0.00 | 5.78 | 9.36 |
| Control sample-3 (inappropriate) | 0.00 | 6.95 | 11.62 |
| Example sample-1 (disclosed sample) | 0.00 | 13.08 | 18.02 |
| Example sample-2 (lower-limit sample) | 0.00 | 11.57 | 16.22 |
| Example sample-3 (upper-limit sample) | 0.00 | 14.63 | 20.44 |

Results: In terms of long-acting hydrating effect, if the disclosed sample is used continuously, there is a process of increasing the water content of the skin, and the water content of the skin will increase by about 18.02% on Day 28. Moreover, it also reflects that the barrier function of the skin is repaired and the water content of the skin is improved.

Compared with Comparative Example 1, Examples 1 to 3 can significantly improve the hydrating (long-acting and instant) effect, indicating that the composition of the present disclosure has a significant efficacy in hydrating (long-acting and instant) ability; p<0.05.

Compared with Comparative Example 2, Examples 1 to 3 have more significant effect in hydrating (long-acting and instant) effect, indicating that the composition of the present disclosure is more reasonable in its component matching, each component is indispensable, and all the components work together to produce a significant synergistic effect.

Compared with Comparative Example 3, Examples 1 to 3 have more significant effect in the hydrating (long-acting and instant) effect, indicating that the composition of the present disclosure has a better effect under the same content of active ingredients. In other words, the components in the composition of the present disclosure are in a reasonable and appropriate ratio.

Among the samples, Example 3 has the most significant effect, indicating that its ratio and concentration are the most suitable, and the effect of the obtained sample is significantly different from the effects of other groups; p<0.05.

3) Barrier Repair Effect on the Skin (TEWL Value)
Test equipment: Multifunctional skin tester with a probe model of Tewameter TM300 provided by CK company, German.
Test principle: FICK's law of diffusion: dm/dt=D·A·dp/dx. Two sets of temperature and humidity sensors were used to measure the water vapor pressure gradient formed by the water loss of the stratum corneum at different bright spots near the epidermis (within about 1 cm), and the transepidermal water loss (TEWL) was directly measured. The TEWL value is an important indicator of the quality of the skin barrier. The lower the TEWL value of the skin, the better the barrier function of the skin, and vice versa.
Test environment: The test environment had a temperature of 22±1° C. and a humidity of 50±5%, and the temperature and humidity were dynamically detected in real time.
Test volunteers: there were at least 30 valid volunteers, aged between 16 and 65 years (except pregnant or lactating women). The valid volunteers were: those who have no serious system disease, no immunodeficiency or autoimmune disease; those who have no history of severe allergies to skin care cosmetics in the past; those who have not used hormone drugs and immunosuppressants in the past month; those who have not participated in other clinical trials; those who have used the test drugs as required and have complete information. All the volunteers should fill out an informed consent form before testing.

Test Steps:

Preparation before testing: No products (cosmetics or topical drugs) were allowed to be applied to the test site 2 to 3 days before testing. Before testing, the subjects were willing to clean inner sides of forearms of both hands and let them air dry naturally. After cleaning, the subjects were marked with the measurement areas on the inner sides of the forearms of both hands. Before the formal testing, the subjects were required to sit still in a standard room for at least 30 min without drinking water, with their forearms exposed, taking a test posture and staying relaxed.

Test process: In the experiment, 3×3 cm² test areas were marked on the inner sides of both the left and right arms. Multiple areas could be marked on the same arm, with a spacing of 1 cm. Both the test products and the blank controls were randomly distributed on the left and right arms. The water loss measurements were performed on the skin in the test and control areas using a probe Tewameter TM300. The measurement was carried out 15 times in parallel on each area. The blank value of each test area was measured first, and then the sample was injected into a piece of film cloth according to a dosage of 0.072 ml of sample/cm², and then the film cloth was applied to the test area for 20 min. 20 min later, the mask cloth was removed. 10 min later, the water loss of the skin in this area was tested, thus obtaining the water loss of the skin at the 30th minute. Then, the water loss of the skin in the test areas and the blank control areas was measured respectively at the 1st hour and the 2nd hour and the test for the same volunteer was done by the same measurement personnel.

For the long-acting barrier repair test, each subject applied the test sample to the same area every day according to the above test method, and on day 14 and day 28, the water loss (TEWL) of the skin was tested without applying the test sample.

Test data: According to the experimental design, the water loss of the skin in each time period was measured, and the reduction in water loss of the skin at each time point was calculated. The greater the reduction in water loss of the skin, the better the skin barrier repair effect.

$$Reduction in water loss of the skin ( \% ) = \frac{( {T}_{0} - {T}_{1} )}{{T}_{0}} \times 100 \%$$

Experimental Results

3.1) Instant Barrier Repair Effect

TABLE 6
Instant reduction in water loss of the skin

| | Reduction in water loss of the skin (%) | | | |
|---|---|---|---|---|
| Time (min) | 0 | 30 | 60 | 120 |
| Control sample-1 | 0.00 | 4.62 | 4.62 | 3.84 |
| Control sample-2 | 0.00 | 6.28 | 6.64 | 6.41 |
| Control sample-3 (inappropriate) | 0.00 | 8.29 | 8.93 | 7.85 |
| Example sample-1 (disclosed sample) | 0.00 | 11.53 | 12.31 | 12.33 |
| Example sample-2 (lower-limit sample) | 0.00 | 11.32 | 11.65 | 10.79 |
| Example sample-3 (upper-limit sample) | 0.00 | 13.53 | 14.68 | 13.76 |

Results: In terms of the reduction in water loss of the skin, the control sample-1, as a basic sample, has a certain effect of reducing the water loss of the skin. At the 30th minute, the reduction of water loss of the skin is 4.62%. For the disclosed sample, the reduction of water loss of the skin reaches 11.53%, which significantly reduces the water loss of the skin, reflecting the effect of skin barrier repair.

3.2) Long-Acting Barrier Repair Effect

TABLE 7
Long-acting reduction in water loss of the skin

| | Reduction in water loss of the skin (%) | | |
|---|---|---|---|
| Time (d) | 0 | 14 | 28 |
| Control sample-1 | 0.00 | 1.93 | 4.84 |
| Control sample-2 | 0.00 | 8.89 | 14.29 |
| Control sample-3 (inappropriate) | 0.00 | 6.93 | 11.71 |
| Example sample-1 (disclosed sample) | 0.00 | 14.40 | 18.93 |
| Example sample-2 (lower-limit sample) | 0.00 | 12.93 | 17.08 |
| Example sample-3 (upper-limit sample) | 0.00 | 18.60 | 21.26 |

Results: In terms of the results of the long-acting reduction in water loss of the skin, the continuous use of Example sample-1 for 28 days results in a reduction of the water loss of the skin of 18.93%; that is, the water loss of the skin is continuously reduced, reflecting that the barrier function of the skin has been effectively repaired.

Compared with Comparative Example 1, Examples 1 to 3 can significantly improve the moisturizing (long-acting and instant) effect, indicating that the composition of the present disclosure has a significant efficacy in moisturizing (long-acting and instant) ability; p<0.05.

Compared with Comparative Example 2, Examples 1 to 3 have more significant effect in moisturizing (long-acting and instant) effect, indicating that the composition of the present disclosure is more reasonable in its component matching, each component is indispensable, and all the components work together to produce a significant synergistic effect.

Compared with Comparative Example 3, Examples 1 to 3 have more significant effect in the moisturizing (long-acting and instant) effect, indicating that the composition of the present disclosure has a better effect under the same content of active ingredients. In other words, the components in the composition of the present disclosure are in a reasonable and appropriate ratio.

Among the samples, Example 3 has the most significant effect, indicating that its ratio and concentration are the most suitable, and the effect of the obtained sample is significantly different from the effects of other groups; p<0.05.

4) Improvement in Skin Glow
Test equipment: Multifunctional skin tester with a model of GL200 provided by CK company, German

Test Principle:

The glow of the skin surface is reflected by direct reflection and scattered reflection of light irradiating the skin surface. A beam of parallel white light generated by a light emitting diode (LED) at a tip of the probe passed through a planar mirror and then directed to the skin surface at an angle of 60°. Part of the light was directly reflected at the same angle and then directed to a receiving sensor through a planar mirror. Another part of the light was scattered by the skin surface and received by another sensor. In this way, the probe GL200 for testing skin glow can not only test the light directly reflected by the skin and related to the glow, but also test the light scattered by the skin.
Test environment: The test environment had a temperature of 22±1° C. and a humidity of 50±5%, and the temperature and humidity were dynamically detected in real time.
Test volunteers: There were at least 30 valid volunteers, aged between 16 and 65 years (except pregnant or lactating women) and their basic values of the skin water content measurement significance of the capacitance method of the forearm test area were between 15 and 100. The valid volunteers were: those who have no serious system disease, no immunodeficiency or autoimmune disease; those who have no history of severe allergies to skin care cosmetics in the past; those who have not used hormone drugs and immunosuppressants in the past month; those who have not participated in other clinical trials; those who have used the test drugs as required and have complete information. All the volunteers should fill out an informed consent form before testing.

Test Steps:

Preparation before testing: No products (cosmetics or topical drugs) were allowed to be applied to the test site 2 to 3 days before testing. Before testing, the subjects were willing to clean inner sides of forearms of both hands and let them air dry naturally. After cleaning, the subjects were marked with the measurement areas on the inner sides of the forearms of both hands. Before the formal testing, the subjects were required to sit still in a standard room for at least 30 min without drinking water, with their forearms exposed, taking a test posture and staying relaxed.

Test process: In the experiment, 3×3 cm² test areas were marked on the inner sides of both the left and right arms. Multiple areas could be marked on the same arm, with a spacing of 1 cm. Both the test products and the blank controls were randomly distributed on the left and right arms. The glow measurements were performed on the skin in the test and control areas using a probe GL200. The measurement was carried out 15 times in parallel on each area. The blank value of each test area was measured first and denoted as G₀, and then the sample was injected into a piece of film cloth according to a dosage of 0.072 ml of sample/cm², and then the film cloth was applied to the test area for 20 min. 20 min later, the mask cloth was removed. The skin was massaged until the sample was completely absorbed. Each subject applied the test sample to the same area every day according to the above test method, and on day 14 and day 28, the glow Gₙ of the skin in corresponding area was tested without applying the test sample.

Test data: According to the experimental design, the skin glow at each time point was measured, and the increase in skin glow at each time point was calculated.

Increase in skin glow (%)=(Gₙ−G₀)/G₀×100%

Experimental Results

TABLE 8
Increase in skin glow

| | Increase in skin glow (%) | | |
|---|---|---|---|
| Time (d) | 0 | 14 | 28 |
| Control sample-1 | 0.00 | 2.37 | 4.74 |
| Control sample-2 | 0.00 | 4.73 | 6.55 |
| Control sample-3 | 0.00 | 5.64 | 9.44 |
| Example sample-1 | 0.00 | 7.76 | 13.68 |
| Example sample-2 | 0.00 | 6.88 | 11.84 |
| Example sample-3 | 0.00 | 9.21 | 15.15 |

Results: In terms of the results of the increase in skin glow, the continuous use of Example sample-1 for 28 days increase the skin glow to 13.68%; that is, the skin glow is improved effectively, reflecting that the barrier function of the skin has been effectively repaired.

Compared with Comparative Example 1, Examples 1 to 3 can significantly increase the skin glow, indicating that the composition of the present disclosure has a significant efficacy in increasing skin glow; p<0.05.

Compared with Comparative Example 2, Examples 1 to 3 have more significant effect in increasing skin glow, indicating that the composition of the present disclosure is more reasonable in its component matching, each component is indispensable, and all the components work together to produce a significant synergistic effect.

Compared with Comparative Example 3, Examples 1 to 3 have more significant effect in increasing skin glow, indicating that the composition of the present disclosure has a better effect under the same content of active ingredients. In other words, the components in the composition of the present disclosure are in a reasonable and appropriate ratio.

Among the samples, Example 3 has the most significant effect, indicating that its ratio and concentration are the most suitable, and the effect of the obtained sample is significantly different from the effects of other groups; p<0.05.

The above are only the preferred embodiments of the present disclosure. It should be pointed out that for those skilled in the art, without departing from the principles of the present disclosure, several improvements and modifications can also be made, and these improvements and modifications should also be regarded as falling within the scope of the present disclosure.

1. A composition, comprising the following components in parts by mass:
0.5 to 2.0 parts of a Leucojum aestivum bulb extract;
0 to 0.5 part of sodium hyaluronate;
0.2 to 1.0 part of a skin conditioner A;
0.2 to 2.0 parts of a skin conditioner B; and
0.1 to 2.0 parts of adenosine;
the skin conditioner A comprising, in percentage by mass:
3% of yeast glycoprotein;
3% of glutamate;
0.55% of valine;
0.55% of threonine;
1.1% of a preservative;
0.05% of a stabilizer; and
the balance of water;
the skin conditioner B comprising, in percentage by mass:
1% of glutamyl aminoethyl imidazole;
0.4% of a preservative; and
the balance of water.

2. The composition according to claim 1, wherein the sodium hyaluronate comprises the following components in percentage by mass:
14.6% of sodium hyaluronate with a molecular weight of 2.1 million;
23.6% of sodium hyaluronate with a molecular weight of 1.3 million; and
61.8% of hydrolyzed sodium hyaluronate.

3. Application of the composition according to claim 1 in preparation of skincare products for night owls.

4. The application according to claim 3, wherein the skincare products have functions of hydrating and moisturizing, restoring skin glow, and promoting cell energy synthesis.

5. A skincare product for night owls, comprising the composition according to claim 1.

6. The skincare product according to claim 5, the composition according to claim 1 has a percentage of is 1% to 7.5% by mass.

7. The skincare product according to claim 5, comprising:
1% to 7.5% of the composition according to claim 1;
9% of a moisturizer;
1% of a penetration enhancer;
0.4% of a cuticle softener;
1% of a preservative; and
the balance of water.

8. The skincare product according to claim 7, wherein
the moisturizer comprises glycerin and 1,3 butanediol at a mass ratio of 1:1;
the penetration enhancer is pentanediol;
the cuticle softener is hydroxyethylpiperazine ethanesulfonic acid;
the preservative is PHL.

9. The skincare product according to claim 7, comprising the following components in percentage by mass:
0.5% to 2.0% of a Leucojum aestivum bulb extract;
0 to 0.5% of sodium hyaluronate;
0.2% to 1.0% of a skin conditioner A;
0.2% to 2.0% of a skin conditioner B;
0.1% to 2.0% of adenosine;
8% of glycerol;
1% of 1,3 butanediol;
1% of pentanediol;
0.4% of hydroxyethylpiperazine ethanesulfonic acid;
1% of PHL; and
the balance of water;
the skin conditioner A comprising, in percentage by mass:
3% of yeast glycoprotein;
3% of glutamate;
0.55% of valine;
0.55% of threonine;
1.1% of a preservative;
0.05% of a stabilizer; and
the balance of water;
the skin conditioner B comprising, in percentage by mass:
1% of glutamyl aminoethyl imidazole;
0.4% of a preservative; and
the balance of water.

10. A method of preparing the skincare product according to claim 5, comprising:
adding hydroxyethylpiperazine ethanesulfonic acid, sodium hyaluronate, and adenosine to deionized water, heating to 75° C. to 80° C., and stirring and dissolving at 250 r/min to 300 r/min for 20 min;
adding glycerol, 1,3-butanediol, and pentanediol, and stirring and dissolving at 300 r/min for 15 min with a temperature maintaining at 65° C. to 75° C.; and
adding a Leucojum aestivum bulb extract, a skin conditioner A, a skin conditioner B, and PHL, and stirring at 100 r/min for 15 min with a temperature maintaining at 35° C. to 45° C., and then cooling to room temperature to prepare the skincare product.
